(19) United States
(12) Patent Application Publication
(10) Pub. No.: US 2014/0271913 A1
(43) Pub. Date: Sep. 18, 2014

(54) COMPOSITIONS AND METHODS FOR MANUFACTURING SOL-GEL DERIVED BIOACTIVE BOROPHOSPHATE GLASSES FOR MEDICAL APPLICATIONS

(71) Applicants: Gregory J. Pomrink, Newberry, FL (US); Zehra Tosun, Gainesville, FL (US); Cecilia Cao, Gainesville, FL (US); Roy Layne Howell, Gainesville, FL (US)

(72) Inventors: Gregory J. Pomrink, Newberry, FL (US); Zehra Tosun, Gainesville, FL (US); Cecilia Cao, Gainesville, FL (US); Roy Layne Howell, Gainesville, FL (US)

(21) Appl. No.: 14/204,839

(22) Filed: Mar. 11, 2014

Related U.S. Application Data
(60) Provisional application No. 61/782,728, filed on Mar. 14, 2013.
(60) Provisional application No. 61/786,991, filed on Mar. 15, 2013.

(51) Int. Cl. A61K 33/42

(52) U.S. Cl. CPC A61K 33/42
USPC 424/602

(57) ABSTRACT

A sol-gel bioactive glass precursor, method for making sol-gel glasses, resultant sol-gel bioactive glasses, and methods of use thereof which include at least 5 weight percent CaO, at least 10 weight percent P₂O₅, at least 10 weight percent Na₂O, and at least 25 weight percent B₂O₃, wherein the bioactive glass is substantially silica-free. Medical and industrial uses of such glasses.

### RELATED APPLICATIONS

The present patent document claims the benefit of the filing date under 35 U.S.C. §119(e) of Provisional U.S. Patent Application Ser. No. 61/782,728, filed Mar. 14, 2013 and Provisional U.S. Patent Application Ser. No. 61/786,991, filed Mar. 15, 2013, which are hereby incorporated by reference in their entirety.

### BACKGROUND

This invention relates generally to novel sol-gel derived bioactive glasses containing sodium and uses thereof.

Sol-gel processes for making, bioactive glass using sol-gel technology are generally known. For example, U.S. Pat. No. 5,074,916 (the “'916 patent”), the subject matter of which is incorporated herein by reference, discloses sol-gel processing techniques used to produce alkali-free bioactive glass compositions based on SiO₂, CaO₂ and P₂O₅. The '916 patent discloses that by varying the SiO₂ content a range of hydroxyapatite production rates can be obtained. Also, varying the time of exposure to actual or simulated in vivo solutions permits use of a range of allowable proportions of SiO₂. The sol-gel derived compositions disclosed in the '916 patent can be chosen to achieve target values for a thermal expansion coefficient, elastic modulus and volume electrical resistivity. Methods of manufacturing near equilibrium dried sol-gel bioactive glasses are described in U.S. Pat. No. 6,171,986 herein incorporated by reference in its entirety.

The '916 patent explains that one of the advantages of sol-gel derived bioactive glasses over melt derived, is that the use of alkali metal oxides such as Na₂O can be avoided in sol-gel derived bioactive glasses. Such alkali metal oxides serve as a flux or aid in melting or homogenization. The '916 patent points out that the presence of alkali metal oxide ions results in a high pH at the interface between the glass and surrounding fluid or tissue in vivo, and that this can induce inflammation and shut down repair. The '916 patent avoids such issues by using only SiO₂, CaO₂ and P₂O₅ and eliminating the traditional need for sodium or other alkali metal compounds to assist in producing bioactivity.

Patent Application Publication U.S. 2009/0208428 states that the presence of the alkali metals, sodium and potassium, at high concentrations in the bioactive glasses can reduce the usefulness of the bioactive glass in vivo. The preferred sol-gel derived glass disclosed in U.S. 2009/0208428 includes strontium and is alkali-metal free.

Bioglass, melt-derived with code name 45S5, contains 45% SiO₂ in weight percent with 24.5% CaO, 24.5% Na₂O and 6% P₂O₅, and provides a rapid biological response, or in other words, fast bioactivity, when implanted in living tissue as compared to other bioactive glass formulations.

It has been well recognized that the surface reactivity of Bioglass is attributed to its bioactivity. In the early of 1990s, sol-gel bioactive glasses have been reported with higher specific surface area from their porous structure. Since then, 49S, 58S, 68S, 77S, 86S sol-gel compositions have been reported with corresponding 50%, 60%, 70%, 80% and 90% SiO₂ in mole percent, respectively. The specific surface area of all of these compositions is more than 100 times greater than melt-derived 45S5 Bioglass. These compositions typically do not contain Na₂O due to the difficulty in incorporating the Na₂O into the glass network.

Some hemostasis products used worldwide, such as Zeolite and starch powders derived products, owe their hemostatic effect to high specific surface area. It is believed that materials with high surface area adsorb water from the blood rapidly and concentrate clotting proteins and platelets to promote instantaneous clot formation. Sol-gel bioactive glasses possess much higher specific surface area, and should be ideal hemostasis materials in addition to their recognized properties of enhancing bone growth, soft tissue growth and healing as well as oral care in applications such as tooth desensitization, anti-gingivitis and tooth whiting. U.S. Patent Application Publication Nos. 2009/0186013 and 2009/0232902, herein incorporated by reference in their entirety, claim that sol-gel made bioactive silica gel with porous structure and high specific surface area, possessed hemostatic effect. But all of the silica gels reported were made from Si, Ca and P precursors or their inorganic compounds and none of silica gels were reported with a sodium precursor.

Newport et al., “Sol-gel synthesis and structural characterization of P₂O₅—B₂O₃—Na₂O glasses for biomedical applications” J. of Materials Chemistry 19, 150-158 (2009), describes a method to create sol-gel derived borophosphate glasses for biomedical applications, as a lower energy cost alternative with higher purity and better homogeneity of final products. Newport et al. states that very limited exploration has been undertaken on the sol-gel synthesis of phosphate glasses containing B₂O₃ and it is not straight forward. This method extends sol-gel preparation of amorphous borophosphate systems having P₂O₅ as a main component. The compositions are described as silica-free or alumina-free with P2O5 as the main component. Although borophosphate glasses are usually obtained by a conventional melt quenching technique, the authors state that glasses in the system 40(P₂O₅)-x(B₂O₃)-(60-x)(Na₂O)(10≦x≦25 mol %) can be prepared by the sol-gel technique. A mixture of mono- and diethylenephosphates was used as a precursor for P₂O₅; boric acid, and sodium methoxide were used as source compounds for B₂O₃ and Na₂O, respectively. The dried gels obtained were heat treated at 200, 300 and 400° C. Systems with x=20 and x=25 mol % were amorphous up to 400°, whereas systems with B₂O₃ content were partially crystalline. These glasses can be used as degradable temporary implants, in order to promote healing or growth of the surrounding tissue, as well as alleviating the need for secondary surgery to remove the implant. Newport et. al. also suggests that these glasses may find applications in drug delivery systems.

### SUMMARY

In one aspect the present invention is directed to a sol-gel derived bioactive glass composition, wherein the bioactive glass is at least 5 weight percent CaO, at least 10 weight percent P₂O₅, at least 10 weight percent Na₂O, and at least 25 weight percent B₂O₃, wherein the bioactive glass is substantially silica-free. The bioactive glass may have a granular form, particulate form, matt form, fiber form, hemostatic sponge form, foam form, paste or putty form, or sphere or bead form, or a combination thereof.

In another aspect, the present invention is directed to a sol-gel bioactive glass precursor including a source of Ca, P, Na, and B wherein the sol-gel bioactive glass precursor is substantially Si free.

In yet another aspect, the present invention is directed to a method of making a sol-gel bioactive glass, wherein the bioactive glass is at least 5 weight percent CaO, at least 10 weight percent P₂O₅, at least 10 weight percent Na₂O, and at least 25 weight percent B₂O₃, wherein the bioactive glass is substantially silica-free comprising: mixing a sol-gel bioactive glass precursor including a source of B, Ca, P, and Na; aging the mixture, and; drying the mixture to form the sol-gel bioactive glass. Also provided by the invention is a method of stimulating osteoblast differentiation and/or proliferation. In the method, the sol-gel derived bioactive glass composition is contacted with an osteoblast. The sol-gel derived bioactive glass composition releases ions and is effective to induce osteoblast differentiation and/or proliferation. Further, the ions released by the sol-gel derived bioactive composition are effective to promote hemostasis and to speed wound healing.

In another aspect, the present invention is directed to a method of inducing rapid coagulation or hemostasis in a bleeding patient comprising contacting the patient with the sol-gel bioactive glass.

In another aspect, the invention relates to a method of making a sol-gel bioactive glass, wherein the bioactive glass is at least 5 weight percent CaO, at least 5 weight percent P₂O₅, at least 10 weight percent Na₂O, and at least 25 weight percent B₂O₃, wherein the bioactive glass is substantially silica-free. The method includes mixing a sol-gel bioactive glass precursor including a source of B, Ca, P, and Na; aging the mixture, and drying the mixture to form the sol-gel bioactive glass.

Other compositions, methods, features and advantages of the invention will be, or will become, apparent to one with skill in the art upon examination of the following figures and detailed description. It is intended that all such additional systems, methods, features and advantages be within the scope of the invention, and be encompassed by the following claims.

### DETAILED DESCRIPTION OF THE PREFERRED EMBODIMENTS

Unless defined otherwise, all technical and scientific terms used herein have the same meaning as commonly understood by one of ordinary skill in the art to which this invention pertains.

A sol-gel bioactive glass, bioactive glass precursor, method for making sol-gel glasses, and resultant sol-gel bioactive glasses are disclosed herein which include Ca, P, Na, and B wherein the sol-gel bioactive glass precursor is substantially Si free. The precursor includes organometallic or inorganic salts of Ca, P, Na, and B that are converted to their respective oxides after heat treatment. The resultant gels provide a homogenous material. This gel may be heat treated at relatively low temperature of 100° C. or less to preserve the porous structure with a high specific surface area thereby avoiding a sintering step and providing the possibility of adding biologically active molecules such as disclosed in U.S. Pat. No. 5,830,480, the contents of which is hereby incorporated by reference in its entirety. The sol-gel glasses are optionally sintered at 500-1000° C. or preferably 550-650° C. It is expected that bioactive sol-gels made in accordance with the present invention will provide significantly improved hemostatic properties as compared to melt-derived 45S5 Bioglass, and other sol-gel compositions. In addition, it is expected that bioactive sol-gels made in accordance with the present invention will exhibit equivalent or better hemostatic properties as compared to some current commercially available hemostasis products.

A sol-gel bioactive glass precursor in accordance with the present invention is a mix of ingredients that provide sources of Ca, P, B, and Na. The precursor is substantially free of silica. “Substantially free of silica” is intended to mean trace amounts naturally present in other substances typically incorporated into sol-gel bioactive glasses or naturally present in the environment in which such glasses are made or used. Many organometallic compounds or inorganic salts providing a source of Ca, P, B, or Na can be used. For example, calcium methoxyethoxide may be used as a source of calcium, triethylphoshpate may be used as a source of phosphorous, triisopropylborate may be used as a source of boron, and sodium ethoxide may be used as a source of sodium. Sol-gel bioactive precursors and sol-gels made therefrom may further contain K, Mg, Zn, B, F, Ag, Cu, Fe, Mn, Mo, Sr, and Zn.

The sol gel bioactive glass may further contain sodium. Many organosodium or inorganic sodium salts may be used as a sodium precursor including but not limited to sodium chloride or sodium ethoxide. Such precursors may be used in an amount sufficient to yield 0-40%, 1-55%, 5-15%, 25-30%, or about 10% by weight Na₂O in the bioactive sol gel glass.

The sol-gel bioactive glass may further comprise potassium. Potassium precursors may include but are not limited to organopotassium compounds or inorganic potassium salts such as potassium nitrate (KNO₃), potassium sulphate (K₂SO₄) and potassium silicates. It is advantageous to provide a bioactive glass composition in which the potassium content is low. If a potassium precursor is included, it may be present in amounts sufficient to yield 0-8 K₂O in the bioactive glass.

The bioactive glass of the present invention preferably comprise calcium. Calcium precursors include but not limited to organocalcium compounds or inorganic salts of calcium such as calcium nitrate (Ca(NO₃)₂), calcium nitrate tetrahydrate (CaNo₃.4H₂O), calcium sulphate (CaSO₄), calcium silicates or a source of calcium oxide. The calcium precursor may be present in the precursor in an amount sufficient to yield at least 5%, 0-40%, 10-20%, 20-30% or about 25% CaO in the resultant sol-gel glass.

The bioactive glass of the present invention preferably comprises P₂O₅. Phosphate precursors include many organophosphates and inorganic phosphate salts including but not limited to triethylphosphate. Release of phosphate ions from the surface of the bioactive glass aids in the formation of hydroxycarbonated apatite. While hydroxycarbonated apatite can form without the provision of phosphate ions by the bioactive glass, as body fluid itself contains phosphate ions, the provision of phosphate ions by the bioactive glass increases the rate of formation of hydroxycarbonated apatite. The phosphate precursor may be present in an amount sufficient to yield 0-80%, 0-50%, 20-60%, 20-30%, 25-30%, or about 25% P₂O₅ in the resultant glass.

The sol-gel bioactive glass of the present invention may comprise zinc. Zinc precursors include but are not limited to organozinc compounds or inorganic salts containing zinc such as zinc nitrate (Zn(NO₃)₂), zinc sulphate (ZnSO₄), and zinc silicates and any such compounds that decompose to form zinc oxide. When present, the zinc precursor should be present in amounts sufficient to yield 0.01-5% ZnO in the glass.

The bioactive glass of the present invention may comprise magnesium. Magnesium precursors include but are not limited to organomagnesium compounds or inorganic magnesium salts such as magnesium nitrate (Mg(NO₃)₂), magnesium sulphate (MgSO₄), magnesium silicates and any such compounds that decompose to form magnesium oxide. When included the magnesium source should be present in an amount sufficient to yield 0.01 to 5% MgO in the bioactive glass.

The sol-gel bioactive glass of the present invention also includes boron. The boron precursors include but are not limited to organoborate compounds, inorganic borate salts, boric acid, and trimethyl borate. A sufficient amount of boron precursor may be used sufficient to provide B₂O₃ in amounts of at least 25%, 30% to 50%, 35-45%, or up to 80% by weight in the glass.

The bioactive glass of the present invention may comprise fluorine. Fluorine precursors include but are not limited to organofluorine compounds or inorganic fluorine salts such as calcium fluoride (CaF₂), strontium fluoride (SrF₂), magnesium fluoride (MgF₂), Sodium fluoride (NaF) or potassium fluoride (KF). Fluoride stimulates osteoblasts, and increases the rate of hydroxycarbonated apatite deposition. When present, an amount of fluorine precursor is used to provide 0-35% or 0.01-5% calcium fluoride.

The bioactive sol-gels may further comprise sources of Si, Cu, Fe, Mn, Mo, or Sr. When present, such sources include organometallic and inorganic salts thereof. Each may be present to provide in 0.01 to 5% or more by weight of the respective oxide in the glass.

Bioactive sol-gels in accordance with the present invention are hemostatic materials that are bioabsorbable, that provide for superior hemostasis, and may be fabricated into a variety of forms suitable for use in controlling bleeding from a variety of wounds, both internal and external. Bioactive sol-gel glasses may be in granular or particulate form, matt or fiber form, a hemostatic sponge, incorporated into a foam, or in the form of a paste or putty. The sol-gel glasses may also be in a form of a sphere or a bead. Exemplary spherical forms were described in U.S. Provisonal Application No. 61/786,991, filed Mar. 15, 2013, content of which is incorporated by reference in its entirety. They may also be formulated into settable and non-settable carriers.

Sol-gel bioactive glass is suitable for use in both surgical applications as well as in field treatment of traumatic injuries. For example, in vascular surgery, bleeding is particularly problematic. In cardiac surgery, the multiple vascular anastomoses and cannulation sites, complicated by coagulopathy induced by extracorporeal bypass, can result in bleeding that can only be controlled by topical hemostats. Rapid and effective hemostasis during spinal surgery, where control of osseous, epidural, and/or subdural bleeding or bleeding from the spinal cord is not amenable to sutures or cautery, can minimize the potential for injury to nerve roots and reduce the procedure time. In liver surgery, for example, live donor liver transplant procedures or removal of cancerous tumors, there is a substantial risk of massive bleeding. An effective hemostatic material can significantly enhance patient outcome in such procedures. Even in those situations where bleeding is not massive, an effective hemostatic material can be desirable, for example, in dental procedures such as tooth extractions, as well as the treatment of abrasions, burns, and the like. In neurosurgery, oozing wounds are common and are difficult to treat.

The bioactive sol-gels may be further combined with a bioactive agent. The bioactive agent comprises one of antibodies, antigens, antibiotics, wound sterilization substances, thrombin, blood clotting factors, conventional chemo- and radiation therapeutic drugs, VEGF, antitumor agents such as angiostatin, endostatin, biological response modifiers, and various combinations thereof. The bioactive sol-gels may also be combined with polymers to provide further structural support. For example, porous bioactive glass hemostatic agents may be prepared by a sol gel process described herein that further uses a block copolymer of ethyleneoxide and propyleneoxide.

Other uses for the sol-gel compositions of the present invention include filling bone defects, bone repair/regeneration, limb salvage, drug delivery, repair of osteochondral defects, reparing osseous defects, dental hypersensitivity, tooth whitening, and guided tissue regeneration.

### EXAMPLES

### Preparation of Sol-Gels

### Example 1

Preparation of Borophosphate sol-gel glass: 20 grams of 100% ethanol (Sigma-Aldrich), 32 grams of triisopropylborate (Sigma-Aldrich 98%), 6 grams of triethylphosphate (Sigma-Aldrich) and 4 grams of 1N nitric acid (Sigma-Aldrich) are added to a glass beaker with a stir bar. The contents are mixed at medium speed and allowed to react for 30 minutes. Then, 50 grams of calcium methoxyethoxide (Gelest 20% in solution) and 81 grams of sodium ethoxide (Sigma-Aldrich 21% in solution) are added to the mixture to yield a sol gel borate solution.

20 grams of 100% Ethanol and 20 grams of 1N nitric acid are mixed together separately and then added dropwise to the sol gel borate solution prepared above with rapid stirring for 30 minutes. Then, the stir bar is removed and a cap is placed on the beaker. The capped beaker is placed in an oven at 60° C. for 24 hours to complete the reaction. The cap is them removed and the mixture allowed to dry at 90° C. in an oven for three days. After the phosphate rich-borate gel is dry, it is placed in a furnace and sintered at 500° C. for three hours.

The above procedure will yield a bioactive glass that is approximately 20 wt % CaO, 15 wt % P₂O₅, 25 wt % Na₂O and 40 wt % B₂O₃.

### Example 2

Preparation of sol-gel Borophosphate Glass: 25 grams of triethylphosphate (Sigma-Aldrich) and 4 grams of 1N Nitric acid (Sigma-Aldrich) are added to a glass beaker with a stir bar. The contents are then mixed and allowed to react at medium speed for 30 minutes. Then, 38 grams of triisopropylborate (Sigma-Aldrich 98%), 24 grams of calcium methoxyethoxide (Gelest 20% in solution), and 40 grams of sodium ethoxide (Sigma-Aldrich 21% in solution) are added, followed by 30 more minutes of mixing.

20 grams of 100% ethanol and 9 grams of 1N nitric acid are mixed together separately and then added dropwise to the sol gel borate solution prepared above with stirring until gelation occurs. Then, the stir bar is removed and a cap is placed on the beaker. The capped beaker is placed in an oven at 60° C. for 24 hours to complete the reaction. The cap is them removed and the mixture allowed to dry at 90° C. in an oven for three days. After the phosphate rich-borate gel is dry, it is placed in a furnace and sintered at 500° C. for three hours.

This procedure will yield a bioactive glass that is approximately 10 wt % CaO, 40 wt % P₂O₅, 15 wt % Na₂O and 35 wt % B₂O₃.

Upon testing glasses made in accordance with Examples 1 and 2, it is believed that such glasses will have a much higher rate of conversion (3 or 4 times faster) than glasses containing silica.

Another aspect of the invention provides for a method of stimulating the activity of a gene that promotes wound healing and/or bone regeneration. Bioactive glass is applied to the site at or near the bone defect. The bioactive glass may be in the form of a particle, a glass sheet, a fiber, a mesh, or any combination of these forms. The activity of the gene is stimulated.

In various embodiments of this aspect, the gene may be one or more of BMP-2, Runx2, Osterix, DIx5, TGF-beta, PDGF, VEGF, collagen I, ALP (alkaline phosphatase), bone sialoprotein, P1NP (procollagen type 1 N-terminal propeptide), osteoponin, osteonectin, and osteocalcin.

BMP-2, also known as bone morphogenetic protein 2, is a member of the TGF-beta superfamily of proteins. Stimulation of BMP-2 activity, such as by stimulating the BMP-2 gene and/or protein expression, can lead to stimulation of bone production. BMP-2 stimulation may enhance the overall rate and extent of bone defect repair.

Runx2, also known as Runt-related transcription factor 2, is a transcription factor that is associated with osteoblast development and differentiation. Mutations in the Runx2 gene are associated with Cleidocranial dysostosis, a general skeletal condition. Stimulation of Runx2 activity, such as by stimulating the Runx2 gene and/or expression of its associated protein, can lead to stimulation of bone production. Runx2 stimulation may enhance osteoblast formation and activity, as well as the overall rate and extent of bone defect repair.

Osterix is a transcription factor that plays a role in osteoblast differentiation and bone formation. As discussed in Cao et al., Cancer Res., 2005, 65:1124-8, Osterix may play a role in osteoblast differentiation and tumor activity in osteosarcoma. Stimulation of Osterix activity, such as by stimulating the Osterix gene and/or protein expression, can lead to stimulation of bone production. Osterix stimulation may enhance the overall rate and extent of bone defect repair.

DLX-5 is a protein that is encoded by the homeobox transcription factor gene DLX5. Mutations in DLX-5 may be associated with hand and foot malformations. Stimulation of DLX-5 protein expression and/or activity, as well as stimulation of DLX5 gene expression, may lead to stimulation of bone production and enhancement of bone defect repair.

TGF-beta (transforming growth factor beta) is a protein that exists in three isoforms, TGF-beta1, TGF-beta2, and TGF-beta3. Genes encoding these proteins include TGFB1, TGFB2, and TGFB3. Activation of these genes, as well as enhancement of the activity of the TGF-beta proteins, can promote tissue remodeling. Increased tissue remodeling can serve to enhance the rate of tissue repair and wound healing.

PDGF (platelet-derived growth factor) is a growth factor that regulates cell growth and division. PDGF plays a major role in angiogenesis, as well as cell proliferation, cell migration, and embryonic development. GAGs may serve to increase PDGF activity as a means to promote wound healing by any one or more of these mechanisms. PDGF is found as four ligands, PDGFA, PDGFB, PDGFC, and PDGFD. These ligands may form dimers. Also, PDGFA and PDGFB may form a heterodimer.

VEGF (vascular endothelial growth factor) is a family of growth factors that include VEGF-A, VEGF-B, VEGF-C, VEGF-D, and PGF (placenta growth factor). VEGF stimulates angiogenesis and promotes cell migration, both processes useful in the repair of soft-tissue wounds. GAGs may promote VEGF-mediated activity. Further, in various embodiments of any aspect of the invention, drugs such as bevacizumab and ranibizumab, which enhance VEGF activity, may be included in the GAG-bioactive glass compositions.

Collagen I, also known as type-I collagen, is found both in scar tissue and in the organic part of bone. Collagen I is also found in tendons and the endocmysium of myofibrils. Stimulation of collagen I production, such as by stimulating expression of genes associated with collagen I, including COL1A1 and COL1A2, may enhance the overall rate and extent of bone defect repair.

ALP, also known as ALKP and alkaline phosphatase, removes phosphate groups from many types of molecules. ALPL, an alkaline phosphatase isozyme, is found in various tissues of the human body, including bone. Stimulation of ALP and/or ALPL activity, may lead to stimulation of bone production. ALP and/or ALPL stimulation may enhance the overall rate and extent of bone defect repair.

Bone sialoprotein, also known as BSP, cell-binding sialoprotein or integrin-binding sialoprotein, is a significant component of bone extracellular matrix. The IBSP gene encodes bone sialoprotein. Stimulation of IBSP gene expression and/or bone sialoprotein expression, may enhance the overall rate and extent of bone defect repair. For example, bone sialoprotein could improve the mineralization of newly-formed bone matrix at the repair site.

Procollagen type 1 N-terminal propeptide, also known as P1NP, is an effective marker of bone formation as this gene promotes collagen turnover. P1NP expression is proportional to the amount of new collagen laid down when bone is formed. Stimulation of P1NP gene expression and/or P1NP protein expression, may enhance the overall rate and extent of bone defect repair by enhancing the rate of collagen deposition in the bone.

Osteopontin, also known as BSP-1, ETA-1, SPP1, 2ar, and Ric, is a protein expressed in bone, as well as other tissues. Ostepontin is synthesed by fibroblasts, preosteoblasts, osteoblasts, osteocytes, bone marrow cells, and endothelial cells. Osteopontin is known to be important in bone remodeling, such as by anchoring osteoclasts to the bone mineral matrix. Stimulation of osteopontin gene expression and/or osteopontin protein expression, may enhance the overall rate and extent of bone defect repair by enhancing the rate of bone formation

Osteonectin, also known as SPARC or BM-40, is a protein encoded by the SPARC gene. Osteonectin binds sodium and is secreted by osteoblasts during bone formation. Osteonectin is thought to play an important role in bone mineralization and collagen binding. As high levels of osteonectin are detected in active osteoblasts, stimulation of SPARC gene expression and/or osteonectin protein expression may enhance the overall rate and extent of bone defect repair by enhancing the rate of bone formation.

Osteocalcin, also known as BGLAP, is a bone protein encoded by the BGLAP gene. Osteocalcin is secreted by osteoblasts and may play a role in bone mineralization. Stimulation of osteocalcin protein expression and/or BGLAP gene expression may enhance the overall rate and extent of bone defect repair.

In some aspects, a compound bone fracture may be treated. A bone at the site of the compound bone fracture is wrapped with any of the above-described compositions of bioactive glass coated with glycosaminoglycans. The bioactive glass ceramic may be in the form of fibers, a fiber mesh, and a sheet. The compositions may have enhanced anti-inflammatory activities that serve to reduce pain and discomfort in the surrounding wounded tissue as the compound bone fracture heals.

The coated bioactive glass fibers, meshes, and sheets may be wrapped completely around the bone such that the ceramic is secured to the bone and/or maintains the bone shape so as to prevent further fracturing. One exemplary form of the bioactive glass ceramic is in the form of a mesh that can be wrapped around a large portion of bone surrounding the compound fracture so as to both provide pressure to the bone and to allow for the migration of ions from the mesh wrap into the bone. The bioactive glass ceramic may also be secured to the bone by one or more plates and/or one or more screws.

In some aspects of the invention, any of the above-described bioactive glasses derived from sol-gels are applied to a wound. The sol-gel derived bioactive glass composition releases ions into the wound. Ions released are one or more of calcium, phosphate, sodium, and borate. Local sources of magnesium, zinc, strontium, silver, zinc and other ions that may be included in and/or released by bioactive glass may enhance the rate of wound healing. The presence of additional magnesium and zinc ions, in particular, may serve to signal cells to enhance the rate of wound healing. Silica ions, along with the increased pH arising from release of sodium ions are conducive to wound healing. In addition, articles by Jung et al. indicate that borate ions may promote wound healing. Silver ions may be effective to reduce inflammation and to inhibit bacterial growth. With regard to bone repair, the presence of calcium, borate and phosphate ions at critical concentrations near the bone can activate genes responsible for osteo progenitor cells to differentiate into osteoblasts. See, e.g., Jones, J. R. et al, “Extracellular matrix formation and mineralization on a phosphate-free porous bioactive glass scaffold using primary human osteoblast (HOB) cells” Biomaterials, 2007, 28(9):1653-63.

In some other aspects of the invention, osteoblast differentiation is induced. An osteoblast is contacted with any of the above-described bioactive glasses derived from sol-gels. The sol-gel derived bioactive glass composition releases ions and is effective to induce osteoblast differentiation. Ions released are one or more of calcium, phosphate, sodium, and borate.

In some other aspects of the invention, osteoblast proliferation is induced. An osteoblast is contacted with any of the above-described bioactive glasses derived from sol-gels. The sol-gel derived bioactive glass composition releases ions and is effective to induce osteoblast proliferation. Ions released are one or more of calcium, phosphate, sodium, and borate.

Throughout this specification various indications have been given as to preferred and alternative embodiments of the invention. However, the foregoing detailed description is to be regarded as illustrative rather than limiting and the invention is not limited to any one of the provided embodiments. It should be understood that it is the appended claims, including all equivalents, that are intended to define the spirit and scope of this invention.

1. A sol-gel derived bioactive glass composition, wherein the bioactive glass is at least 5 weight percent CaO, at least 10 weight percent P₂O₅, at least 10 weight percent Na₂O, and at least 25 weight percent B₂O₃, wherein the bioactive glass is substantially silica-free.

2. The sol gel derived bioactive glass composition of claim 1, wherein the bioactive glass has a granular form, particulate form, matt form, fiber form, hemostatic sponge form, foam form, paste or putty form, or sphere or bead form, or a combination thereof.

3. A sol-gel bioactive glass precursor including a source of Ca, P, Na, and B wherein the sol-gel bioactive glass precursor is substantially Si free.

4. The sol-gel bioactive glass precursor of claim 3, wherein the B source is triisopropyl borate.

5. The sol-gel bioactive glass precursor of claim 3, wherein the Ca source is calcium methoxymethoxide.

6. The sol-gel bioactive glass precursor of claim 3, wherein the P source is triethylphosphate.

7. The sol-gel bioactive glass precursor of claim 3, wherein the Na source is NaCl.

8. The sol-gel bioactive glass precursor of claim 3, wherein the Na source is C₂H₅ONa.

9. The sol-gel bioactive glass precursor of claim 3, wherein the source of Na is NaCl and is present in an amount to provide for 20-30% by weight of Na₂O in a sol-gel bioactive glass.

10. The sol-gel bioactive glass precursor of claim 3, wherein the source of Na is C₂H₅ONa and is present in an amount to provide for 20-30% by weight of Na₂O in a sol-gel bioactive glass.

11. The sol gel bioactive glass precursor of claim 3, wherein the source of phosphate is triethylphosphate and is present in an amount to provide for 20-30% by weight of P₂O₅ in a sol-gel bioactive glass.

12. A method of making a sol-gel bioactive glass, wherein the bioactive glass is at least 5 weight percent CaO, at least 10 weight percent P₂O₅, at least 10 weight percent Na₂O, and at least 25 weight percent B₂O₃, wherein the bioactive glass is substantially silica-free comprising:
mixing a sol-gel bioactive glass precursor including a source of B, Ca, P, and Na;
aging the mixture, and;
drying the mixture to form the sol-gel bioactive glass.

13. The method of claim 12, wherein the B source is triisopropyl borate.

14. The method of claim 12, wherein the Ca source is calcium methoxymethoxide.

15. The method of claim 12, wherein the P source is triethylphosphate.

16. The method of claim 12, wherein the Na source is NaCl.

17. The method of claim 12, wherein the Na source is C₂H₅ONa.

18. The method of claim 12, wherein said aging is conducted at a temperature of 50-80° C. for 40-70 hours.

19. The method of claim 12, wherein said drying is conducted at 400-600° C. for 15 to 50 hours.

20. A method for achieving hemostasis in a patient in need of treatment thereof comprising contacting the patient with the sol-gel bioactive glass of claim 1.

21. A method of inducing rapid coagulation in a bleeding patient comprising contacting the patient with the sol-gel bioactive glass of claim 1.

22. A method for achieving hemostasis in a patient in need of treatment thereof comprising contacting the patient with a sol-gel bioactive glass made from the sol-gel bioactive glass precursor of claim 3.

23. The sol-gel derived bioactive glass composition of claim 1, further comprising an extracellular matrix protein.

24. A method of treating a wound comprising applying the sol-gel derived bioactive glass composition of claim 1 to the wound, wherein the sol-gel derived bioactive glass composition releases ions into the wound.

25. A method of stimulating osteoblast differentiation comprising contacting an osteoblast with the sol-gel derived bioactive glass composition of claim 1, wherein the sol-gel derived bioactive glass composition releases ions and the method is effective to induce osteoblast differentiation.

26. A method of stimulating osteoblast proliferation comprising contacting an osteoblast with the sol-gel derived bioactive glass composition of claim 1, wherein the sol-gel derived bioactive glass composition releases ions and the method is effective to induce osteoblast proliferation.

27. A method of repairing bone defects comprising contacting bone in need of treatment thereof with the sol-gel bioactive glass of claim 1.

28. A sol-gel derived bioactive glass composition, wherein the bioactive glass is at least 5 weight percent alkaline earth metal, at least 10 weight percent P₂O₅, at least 10 weight percent alkali metal, and at least 25 weight percent B₂O₃, wherein the bioactive glass is substantially silica-free.

29. The sol-gel derived bioactive glass composition of claim 28, wherein the alkali metal is selected from the group consisting of Na, Li, or K.

30. The sol-gel derived bioactive glass composition of claim 28, wherein the alkaline earth metal is selected from the group consisting of Ca, Mg, Sr or Ba.

31. The sol gel derived bioactive glass composition of claim 28, wherein the bioactive glass has a granular form, particulate form, matt form, fiber form, hemostatic sponge form, foam form, paste or putty form, or sphere or bead form, or a combination thereof.

32. A method of making a sol-gel bioactive glass including Si, Ca, P, and Na comprising:
mixing a sol-gel bioactive glass precursor including a source of Si, Ca, P, and Na, wherein the sodium source is selected from the group consisting of NaCl and C₂H₅ONa, and;
drying the mixture at a temperature of 100° C. or lower.

33. The method of claim 32, further comprising adding a biologically active molecule.

34. A method of making a sol-gel bioactive glass, wherein the bioactive glass is at least 5 weight percent CaO, at least 5 weight percent P₂O₅, at least 10 weight percent Na₂O, and at least 25 weight percent B₂O₃, wherein the bioactive glass is substantially silica-free comprising:
mixing a sol-gel bioactive glass precursor including a source of B, Ca, P, and Na;
aging the mixture, and;
drying the mixture to form the sol-gel bioactive glass.
